(19) United States
(12) Patent Application Publication
(10) Pub. No.: US 2009/0216029 A1
(43) Pub. Date: Aug. 27, 2009

(54) PROCESS FOR THE PRODUCTION OF ATORVASTATIN CALCIUM IN AMORPHOUS FORM

(76) Inventors: Yatendra KUMAR, Gurgaon (IN); Saridi Madhava Dileep Kumar, Gurgaon (IN); Swargam Sathyanarayana, Karim Nager (IN)

Correspondence Address: Jayadeep R. Deshmukh, Esq.;Ranbaxy Inc., Suite 2100, 600 College Road East, Princeton, NJ, 08540, US

(21) Appl. No.: 12/034,838

(22) Filed: Feb. 21, 2008

Related U.S. Application Data
(63) Continuation-in-part of application No. 10/549,890.

(51) Int. Cl. C07D 207/34

(52) U.S. Cl. 548/537

(57) ABSTRACT

A process for the production of amorphous atorvastatin calcium and stabilized, amorphous atorvastatin calcium is provided.

### CROSS REFERENCE TO RELATED APPLICATION

This application is a continuation-in-part of Ser. No. 10/549,890 filed Sep. 16, 2005, the disclosure of the application being incorporated herein by reference.

### FIELD OF THE INVENTION

Processes for the production of atorvastatin calcium of high purity in an amorphous form are provided.

### BACKGROUND OF THE INVENTION

Atorvastatin is known by the chemical name [R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid. The hemi-calcium salt of atorvastatin is useful as an inhibitor of the enzyme 3-hydroxy-3-methylglutaryl-coenzyme A reductase (HMG-CoA reductase) and is thus useful as a hypolipidemic and hypocholesterolemic agent.

U.S. Pat. Nos. 5,273,995; 5,003,080; 5,097,045; 5,103,024; 5,124,482; 5,149,837; 5,155,251; 5,216,174; 5,245,047; 5,248,793; 5,280,126; 5,397,792; and 5,342,952, disclose various processes and intermediates for preparing atorvastatin. Several processes have been reported for the preparation of amorphous form of atorvastatin calcium in U.S. Pat. Nos. 6,528,660 and 6,613,916, U.S. Patent Application Publication Nos. 2002/0183378 and 2003/0109569, and International (PCT) Patent Applications WO 01/02899, WO 02/57228, WO 02/83637, WO 02/83638, WO 03/18547 and WO 03/68739.

### SUMMARY OF THE INVENTION

In one embodiment, a process for the production of atorvastatin calcium in amorphous form is provided comprising:
a) reacting a solution of (4R-cis)-1,1-dimethylethyl-6-{2-[2-(4-fluorophenyl)-5-(1-methylethyl)-3-phenyl-4-[(phenylamino)-carbonyl]-1H-pyrrol-1yl]ethyl}-2,2-dimethyl-1,3-dioxane-4-acetate (Compound H, as shown in Scheme I) in a water miscible solvent with an acid to obtain [R—(R*,R*)]-1,1-dimethylethyl-2-(4-fluorophenyl)-β,δ-dihydroxy-5-(1-methylethyl)-3-phenyl-4-[(phenylamino)carbonyl]-1H-pyrrole-1-heptanoate (Compound I, as shown in Scheme I),
b) treating Compound I with an alkali metal hydroxide to obtain an alkali metal salt of atorvastatin,
c) washing the solution of alkali metal salt of atorvastatin with a solvent immiscible or slightly miscible in water,
d) treating the washed solution of alkali metal salt of atorvastatin with a calcium salt or calcium hydroxide to obtain atorvastatin calcium,
e) isolating crude atorvastatin calcium,
f) purifying crude atorvastatin calcium by dissolving in a mixture of tetrahydrofuran and methanol, and precipitating with water to obtain pure atorvastatin calcium in crystalline form, and
g) converting crystalline pure atorvastatin calcium so obtained into amorphous form.

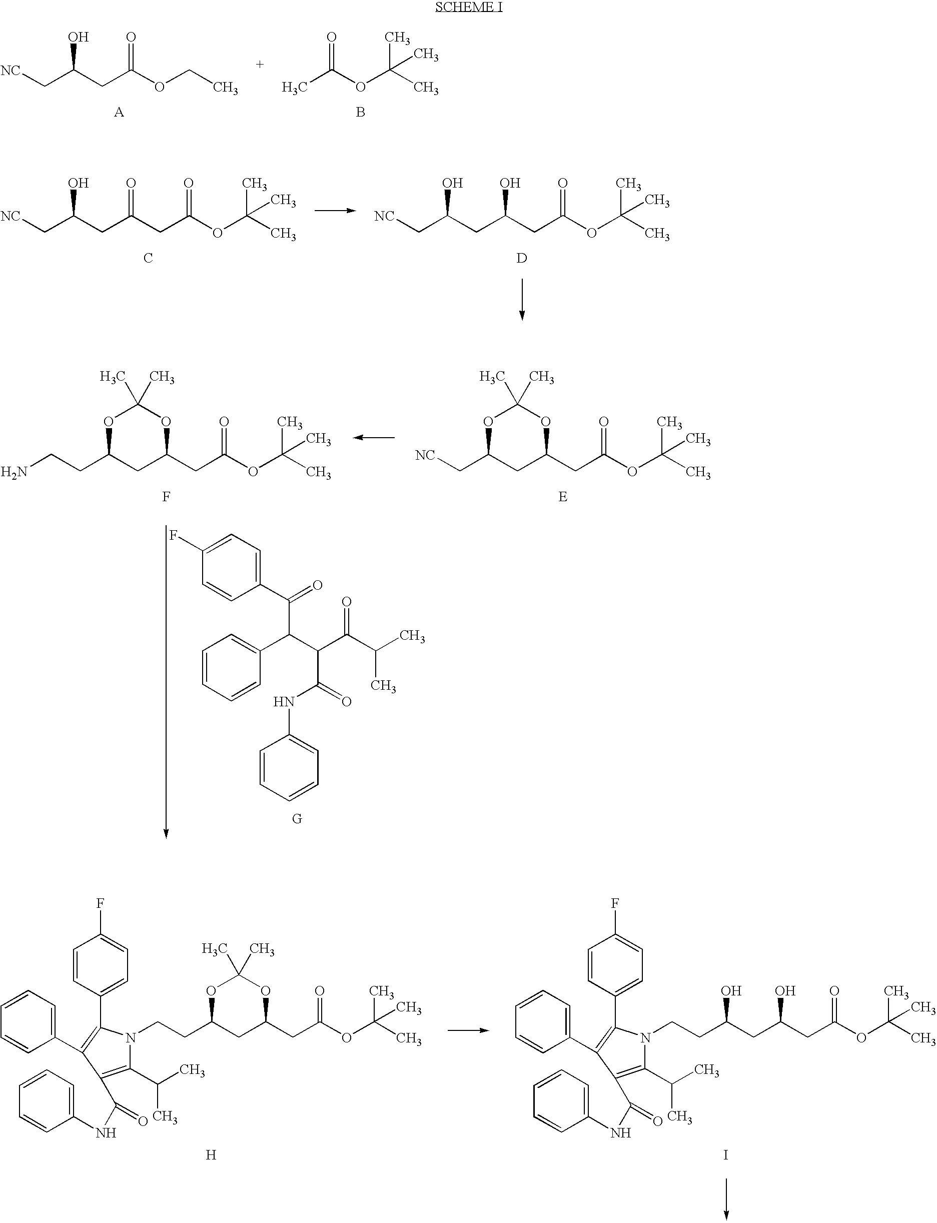

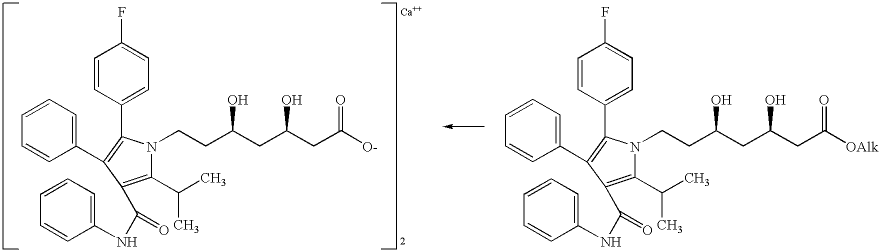

In another embodiment, a process for purifying atorvastatin calcium is provided comprising dissolving crude atorvastatin calcium in a mixture of tetrahydrofuran and methanol, and precipitating with water to obtain pure atorvastatin calcium in crystalline form.

In an additional embodiment, a process for the production of stabilized atorvastatin calcium in amorphous form is provided comprising:
a) dissolving crystalline atorvastatin calcium and an antioxidant in a solvent,
b) adding the solution of atorvastatin calcium and antioxidant to an anti-solvent, and
c) separating precipitated, amorphous atorvastatin calcium from the resulting suspension.

In yet another embodiment, a process for the production of atorvastatin calcium in amorphous form is provided comprising:
a) dissolving crystalline atorvastatin calcium in a hydroxylic solvent,
b) adding the obtained solution of atorvastatin calcium to a non-hydroxylic anti-solvent, wherein the non-hydroxylic anti-solvent has a higher boiling point than the hydroxylic solvent,
c) concentrating the solution so obtained to remove the hydroxylic solvent, and
d) separating precipitated amorphous atorvastatin calcium from the resulting suspension.

The acid used for deketalization of Compound H to afford Compound I may be an inorganic acid. Examples of inorganic acids include hydrochloric, hydrobromic, sulphuric, phosphoric and nitric acids. Suitable water-miscible solvents for the deketalization process include acetonitrile, alcohols such as methanol, ethanol, propanol, and isopropanol, cyclic ethers such as dioxane and tetrahydrofuran, ketones such as acetone and mixtures thereof.

Compound I can be hydrolyzed with an alkali metal hydroxide such as sodium hydroxide, potassium hydroxide and lithium hydroxide. The reaction mixture may be maintained at a pH of at least 9, for example, about 12, to result in efficient hydrolysis and to minimize side product formation. The reaction mixture is then washed with a water-immiscible or slightly water-miscible solvent to remove unreacted compounds and other impurities. Suitable solvents for the washing include ethers such as methyl tertiary butyl ether, diethyl ether, methyl ethyl ether and dibutyl ether, esters such as ethyl acetate and isopropyl acetate, and hydrocarbons such as toluene and petroleum ether.

The solution of alkali metal salt of atorvastatin obtained is reacted with calcium hydroxide or a calcium salt such as calcium acetate, calcium chloride, calcium sulfate, calcium nitrate and calcium phosphate. The reaction may be performed at a temperature of about 45 to 55° C. The pH of the solution of alkali metal salt of atorvastatin may be lowered to about 7.8 to 8.2 with an acid before addition of the calcium salt to facilitate isolation of crude atorvastatin calcium.

Any residual water-immiscible or slightly water-miscible solvent remaining in the reaction mixture may be removed under reduced pressure to aid precipitation. Water may be used as an antisolvent to effect precipitation of crude atorvastatin calcium in good yields. Water may be added at a temperature of about 55 to 65° C. to avoid rapid precipitation and seeds of crystalline atorvastatin calcium may also be added to the mixture. Crude atorvastatin calcium may be isolated in high yields by cooling the reaction mixture to a temperature of about 20 to 35° C. and stirring at the same temperature for several hours before filtration or centrifugation.

Crude atorvastatin calcium is purified by crystallization using tetrahydrofuran and methanol as solvents and water as anti-solvent. Purification involves removal of unreacted compounds, side product and other impurities. Tetrahydrofuran, methanol and water may be used in the volume ratio 1:1:4 to obtain atorvastatin calcium of high purity. Water may be added at a temperature of about 60 to 65° C. Seeds of crystalline atorvastatin calcium may be added to facilitate precipitation. In a particular embodiment, seeds of crystalline atorvastatin calcium are added at a temperature of about 50° C. Crystalline atorvastatin calcium may be isolated by cooling the mixture to a temperature of about 30 to 35° C. and stirring at the same temperature for several hours before filtration or centrifugation.

Crystalline pure atorvastatin calcium may be converted into the amorphous form by methods known in the art such as U.S. Pat. Nos. 6,528,660 and 6,613,916, International (PCT) Patent Applications WO 01/28999, WO 03/99785, WO 03/78379, WO 03/18547 and WO 02/57228, and U.S. Patent Application Publication No. 2002/0183378, which are incorporated herein by reference.

Amorphous atorvastatin calcium may also be obtained by having an additional step wherein the pure crystalline atorvastatin calcium (where “pure” is meant in the sense of chemical purity) obtained after step f) is suspended in a mixture of methanol and water in the volume ratio 1 to 5 and stirred with seed crystals of crystalline Form I, to obtain atorvastatin calcium in crystalline Form I. The stirring may be performed at a temperature of about 10 to 65° C., for example, about 30 to 45° C.

Alternatively, pure crystalline atorvastatin calcium (where “pure” is meant in the sense of chemical purity) obtained after step f) is suspended in a mixture of methanol and water in the volume ratio 3 to 2 and stirred with seed crystals of crystalline Form II, to obtain atorvastatin calcium in crystalline Form II. The volume of methanol and water mixture may be about 15 to 25 times, for example, about 20 times, the weight of the atorvastatin calcium to be suspended. The stirring may be performed at a temperature of about 10 to 65° C., for example, about 25 to 45° C.

In yet another variant, a further additional step may be performed wherein crystalline Form I of atorvastatin calcium obtained above is suspended in a mixture of methanol and water in the volume ratio 3 to 2 and stirred with seed crystals of crystalline Form II, to obtain atorvastatin calcium in crystalline Form II. The volume of methanol and water mixture may be about 15 to 25 times, for example, about 20 times, the weight of the atorvastatin calcium to be suspended. The stirring may be performed at a temperature of about 10 to 65° C., for example, about 25 to 45° C.

Amorphous atorvastatin calcium may be obtained by dissolving crystalline atorvastatin calcium in a solvent, and adding the resulting solution to an anti-solvent. An anti-solvent is a liquid that does not dissolve atorvastatin calcium. Examples of solvents include ketones such as acetone and methyl isobutyl ketone, esters such as ethyl acetate and isopropyl acetate, chlorinated hydrocarbons such as methylene chloride and ethylene dichloride, cyclic ethers such as dioxan and tetrahydrofuran, alcohols such as methanol, ethanol and isopropanol, nitriles such as acetonitrile, dipolar aprotic solvents such as dimethylsulfoxide and dimethylformamide, and mixtures thereof with water. Examples of anti-solvents include hydrocarbons, such as cyclohexane, hexanes, heptanes, petroleum ethers, toluene, xylene and the like, dialkyl ethers such as diethyl ether, diisopropyl ether, and the like, and can readily be determined by one ordinarily skilled in the art.

An antioxidant may be added to the atorvastatin calcium solution to obtain stabilized, amorphous atorvastatin calcium. Examples of suitable antioxidants include butylated hydroxyanisole, butylated hydroxytoluene and tertiary-butylated hydroquinone.

### DETAILED DESCRIPTION OF THE INVENTION

The term ‘stabilized atorvastatin calcium’ means the hemi-calcium salt of atorvastatin having a level of purity, which is provided and maintained through the use of antioxidants.

Stabilized, amorphous atorvastatin calcium can be obtained with purity of at least 97%, for example when determined by high performance liquid chromatography (HPLC) analysis. In general, stabilized, amorphous atorvastatin calcium having a purity of at least 99% may be obtained. In some particular embodiments, stabilized, amorphous atorvastatin calcium having a purity of at least 99.5% may be obtained.

The atorvastatin calcium solution may be dried (moisture removal) before its addition to the non-solubilizing liquid. This may be accomplished by, for example, filtration through dry molecular sieves. Alternatively or additionally, drying of the solution may be achieved by a process, wherein the solution is made using excess solvent, which is then concentrated to remove moisture from the solution.

Examples of hydroxylic solvents which may be used for dissolving atorvastatin calcium include alcohols such as methanol, ethanol, propanol, isopropanol, and mixtures thereof with water. Examples of non-hydroxylic anti-solvents which may have a higher boiling point than the hydroxylic solvent include hydrocarbons, such as cyclohexane, hexanes, heptanes, petroleum ethers, toluene, xylene and the like, dialkyl ethers such as diisopropyl ether, and the like, and can readily be determined by one ordinarily skilled in the art.

The solution of atorvastatin calcium having the desired hydroxylic solvent and non-hydroxylic anti-solvent is concentrated to remove the hydroxylic solvent either partially or completely to precipitate amorphous atorvastatin calcium. In a manner similar to that detailed above, an antioxidant may be added to the hydroxylic solution of atorvastatin calcium to obtain stabilized, amorphous atorvastatin calcium. Similarly, the atorvastatin calcium hydroxylic solution may also be treated as detailed above for moisture removal.

(4R-cis)-1,1-dimethylethyl-6-{2-[2-(4-fluorophenyl)-5-(1-methylethyl)-3-phenyl-4-[(phenylamino)-carbonyl]-1H-pyrrol-1yl]ethyl}-2,2-dimethyl-1,3-dioxane-4-acetate (Compound H, as shown in Scheme I) may be obtained by methods known in the art, such as those described in U.S. Pat. Nos. 5,003,080; 5,103,024; 5,155,251 and Tetrahedron Lett., 33 (17), 2279-82 (1992), which are incorporated herein by reference.

In a particular embodiment, Compound H may be obtained as described in reaction Scheme I by
a) treating (R)-ethyl 4-cyano-3-hydroxybutanoate (Compound A, as shown in Scheme I) with 1,1-dimethylethylacetate (Compound B, as shown in Scheme I) in the presence of n-butyl lithium and diisopropylamine to obtain (R)-1,1-dimethylethyl 6-cyano-5-hydroxy-3-oxohexanoate (Compound C, as shown in Scheme I),
b) treating Compound C with diethyl methoxyborane and sodium borohydride to obtain [R—(R*,R*)]-1,1-dimethylethyl 6-cyano-3,5-dihydroxyhexanoate (Compound D, as shown in Scheme I),
c) treating Compound D with 2,2-dimethoxy propane and methanesulfonic acid to obtain (4R-cis)-1,1-dimethylethyl-[6-cyanomethyl-2,2-dimethyl-1,3-dioxan]-4-acetate (Compound E, as shown in Scheme I),
d) treating Compound E under reducing conditions to obtain (4R-cis)-1,1-dimethylethyl-[6-(2-aminoethyl)-2,2-dimethyl-1,3-dioxan-4-yl]acetate (Compound F, as shown in Scheme I), and
e) condensing Compound F with (±)-4-fluoro-α-(2-methyl-1-oxopropyl)-γ-oxo-N,β-diphenylbenzenebutaneamide (Compound G, as shown in Scheme I) to obtain (4R-cis)-1,1-dimethylethyl-6-{2-[2-(4-fluorophenyl)-5-(1-methylethyl)-3-phenyl-4-(phenylamino)carbonyl]-1H-pyrrol-1yl]ethyl}-2,2-dimethyl-1,3-dioxane-4-acetate (Compound H, as shown in Scheme I).

Crystalline forms of atorvastatin calcium to be used as seeds may be obtained by methods known in the art such as those described in U.S. Publication No. 2002/0183378, which is incorporated herein by reference, or prepared by processes exemplified herein.

A detailed study was carried out to identify the trace impurities resulting from the process, as well as degradation impurities formed under storage or processing in amorphous atorvastatin calcium. Amorphous atorvastatin calcium having a high level of purity is obtained by the processes of the invention. Some samples of amorphous atorvastatin calcium produced were subjected to detailed analysis by HPLC and the following results were observed:

| IMPURITIES OBSERVED IN | | Content found in |
|---|---|---|
| ATORVASTATIN CALCIUM | Content found in | amorphous atorvastatin |
| (RESULTING FROM | amorphous atorvastatin | after storage at 25° C./60% |
| PROCESS OR STORAGE | prepared (% w/w by | relative humidity for 3 |
| DEGRADATION) | HPLC) | months (% w/w by HPLC) |
| Amide impurity of Formula I | Not detected (LOD | Not detected (LOD 0.04%) |
| | 0.041%) to less than 0.05% | to below 0.05% |
| Diastereomer of atorvastatin | Below 0.062(LOQ) to less | Below 0.062(LOQ) to less |
| calcium of Formula II | than 0.5% | than 0.5% |
| Ketal of atorvastatin of | Less than 0.05% to 0.1% | Less than 0.05% to 0.1% |
| Formula III | | |
| 3′-deshydroxy atorvastatin of | Not detected (LOD | Not detected (LOD |
| Formula IV | 0.049%) to less than 0.05% | 0.049%) to less than 0.05% |
| 5′-deshydroxy atorvastatin of | Not detected (LOD | Not detected (LOD |
| Formula V | 0.048%) to less than 0.05% | 0.048%) to less than 0.05% |
| Methyl ester of atorvastatin of | Less than 0.05% | Less than 0.05% |
| Formula VI | | |
| Diepoxide of atorvastatin of | Less than 0.05% | 0.15% to 0.24 |
| Formula VII | | |
| Dihydroxy diepoxide of | Not detected (LOD | Less than 0.05% |
| Formula VIII | 0.035%) to less than 0.05% | |
| Diketoepoxide of Formula IX | Less than 0.05 | Less than 0.20 to 0.33% |
| O-methyl atorvastatin calcium | Less than 0.05% | Less than 0.05% |
| of Formula X | | |
| 3-oxo atorvastatin of Formula | 0.06 to 0.10 | 0.06 to 0.11 |
| XI | | |
| Unidentified Impurity at RRT | Less than 0.05% | Less than 0.07 to 0.11% |
| ~1.03 of MW 590 | | |
| Unidentified impurity at RRT | Less than 0.05% | Less than 0.05 to 0.08% |
| ~1.17 of MW 431 | | |
| Corresponding lactone of | 0.09 to 0.11 | 0.09 to 0.12 |
| atorvastatin of Formula XII | | |
| Atorvastatin calcium of | 0.16 to 0.17 | 0.17 to 0.19 |
| Formula XII | | |

LOD: Limit of detection,
LOQ: Limit of quantification:
RRT: relative retention time

HPLC method:
Column: Kromasil C18, 250×4.6 mm, 5 μm
Mobile phase:
A: 388.5 ml of acetonitrile+31.5 ml of tetrahydrofuran++580 ml of 0.05 molar ammonium dihydrogen orthophosphate solution in water
B: 92.5 ml of acetonitrile+7.5 ml of tetrahydrofuran+300 of methanol++100 ml of 0.05 molar ammonium dihydrogen orthophosphate solution in water
Gradient:

| Time (min) | Flow-rate (ml/min) | A % | B % |
|---|---|---|---|
| 0 | 1.8 | 100 | 0 |
| 20 | 1.8 | 100 | 0 |
| 35 | 1.5 | 25 | 75 |
| 40 | 1.5 | 25 | 75 |
| 55 | 1.5 | 0 | 100 |
| 60 | 1.8 | 100 | 0 |

Injection delay: 10 min
Wavelength: UV at 246 nm
Column oven temperature: 30° C.±1.0° C.
Injected volume: 20 μl
Samples were dissolved in methanol (one part) and diluted with a mixture of acetonitrile:distilled water=2:3, (9 parts) concentration 0.5 mglml the retention time of atorvastatin peak is about 19 min.

| IMPURITIES OBSERVED IN | | |
|---|---|---|
| ATORVASTATIN CALCIUM (RESULTING | RRT1 (w.r.t. | |
| FROM PROCESS OR STORAGE | atorvastatin | |
| DEGRADATION) | peak) | RRT2 |
| Amide impurity of Formula I | ~0.45 | |
| Diastereomer of atorvastatin calcium of | ~0.94 | |
| Formula II | | |
| Ketal of atorvastatin of Formula III | | ~1.27 |
| 3′-deshydroxy atorvastatin of Formula IV | | ~0.98 |
| 5′-deshydroxy atorvastatin of Formula V | | ~1.02 |
| Methyl ester of atorvastatin of Formula VI | | ~1.08 |
| Diepoxide of atorvastatin of Formula VII | | ~1.09 |
| Dihydroxy diepoxide of Formula VIII | | ~1.13 |
| Diketoepoxide of Formula IX | | ~1.19 |
| O-methyl atorvastatin calcium of Formula X | | ~0.89 |
| 3-oxo atorvastatin of Formula XI | ~0.89 | |
| Unidentified Impurity of MW 590 | | ~1.03 |
| Unidentified impurity of MW 431 | | ~1.17 |
| Corresponding lactone of atorvastatin of | | 1.00 |
| Formula XII | | |
| Desfluoro atorvastatin calcium of Formula XIII | 0.84 | |

Formula I

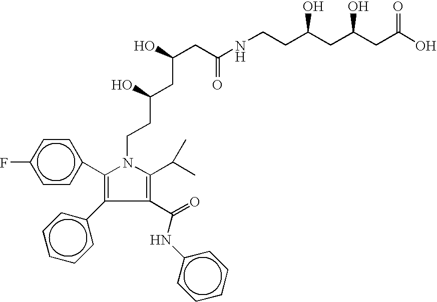

Formula II

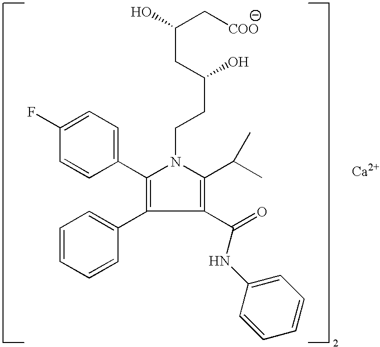

Formula III

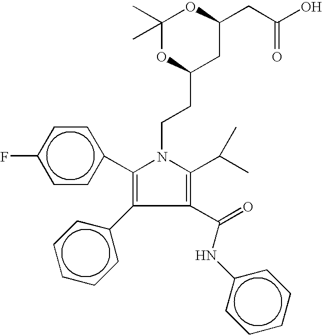

Formula IV

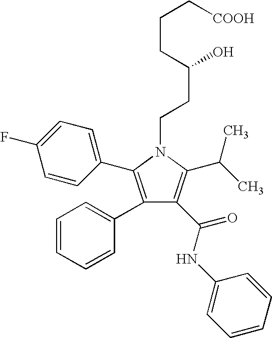

Formula V

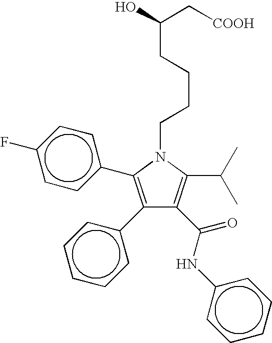

Formula VI

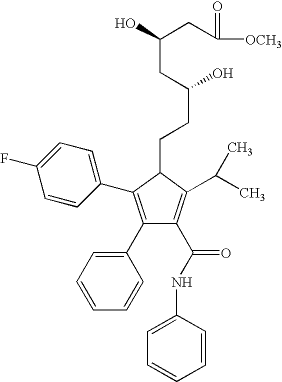

Formula VII

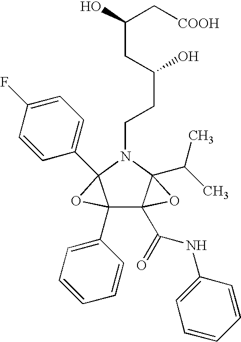

Formula VIII

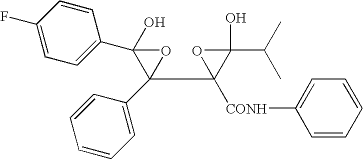

Formula IX

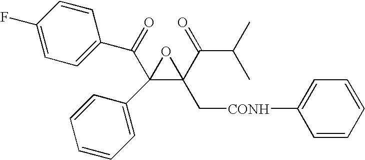

Formula X

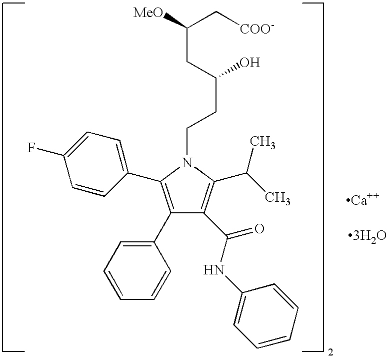

Formula XI

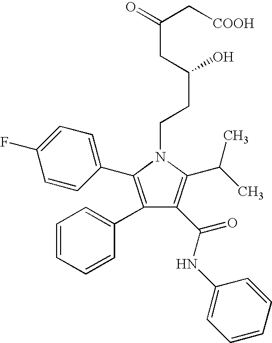

Formula XII

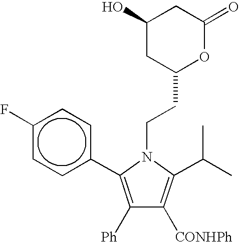

Formula XIII

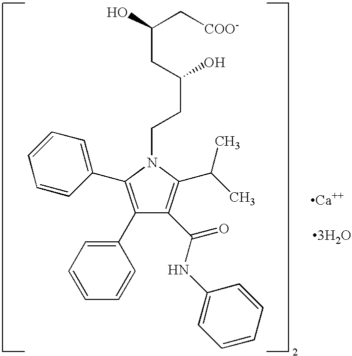

In general, amorphous atorvastatin calcium containing not more than 0.2% of each of the amide impurity of Formula I, diastereomer of atorvastatin calcium of Formula II, ketal of atorvastatin of Formula III, 3′-deshydroxy atorvastatin of Formula IV, 5′-deshydroxy atorvastatin of Formula V, methyl ester of atorvastatin of Formula VI, diepoxide of atorvastatin of Formula VII, dihydroxy diepoxide of Formula VIII, diketoepoxide of Formula IX, O-methyl atorvastatin calcium of Formula X, 3-oxo atorvastatin of Formula XI, unidentified Impurity at RRT˜1.03 of MW 590, or the unidentified impurity at RRT˜1.17 of MW 431 or the corresponding lactone of atorvastatin of Formula XII may be obtained.

Amorphous atorvastatin calcium containing not more than 0.1% of each of the amide impurity of Formula I, diastereomer of atorvastatin calcium of Formula II, ketal of atorvastatin of Formula III, 3′-deshydroxy atorvastatin of Formula IV, 5′-deshydroxy atorvastatin of Formula V, methyl ester of atorvastatin of Formula VI, diepoxide of atorvastatin of Formula VII, dihydroxy diepoxide of Formula VIII, diketoepoxide of Formula IX, O-methyl atorvastatin calcium of Formula X, 3-oxo atorvastatin of Formula XI, unidentified Impurity at RRT˜1.03 of MW 590, unidentified impurity at RRT˜1.17 of MW 431 or the corresponding lactone of atorvastatin of Formula XII may be obtained.

Amorphous atorvastatin calcium containing not more than 0.05% of each of the amide impurity of Formula I, diastereomer of atorvastatin calcium of Formula II, 3′-deshydroxy atorvastatin of Formula IV, 5′-deshydroxy atorvastatin of Formula V, methyl ester of atorvastatin of Formula VI, diepoxide of atorvastatin of Formula VII, dihydroxy diepoxide of Formula VIII, diketoepoxide of Formula IX, O-methyl atorvastatin calcium of Formula X, unidentified Impurity at RRT˜1.03 of MW 590, or the unidentified impurity at RRT˜1.17 of MW 431 may be obtained.

In general, amorphous atorvastatin calcium containing not more than 0.5% of total impurities may be obtained.

Further amorphous atorvastatin calcium obtained contained not more than 0.5% of each of the amide impurity of Formula I, diastereomer of atorvastatin calcium of Formula II, ketal of atorvastatin of Formula III, 3′-deshydroxy atorvastatin of Formula IV, 5′-deshydroxy atorvastatin of Formula V, methyl ester of atorvastatin of Formula VI, diepoxide of atorvastatin of Formula VII, dihydroxy diepoxide of Formula VIII, diketoepoxide of Formula IX, O-methyl atorvastatin calcium of Formula X, 3-oxo atorvastatin of Formula XI, unidentified Impurity at RRT˜1.03 of MW 590, unidentified impurity at RRT˜1.17 of MW 431 or the corresponding lactone of atorvastatin of Formula XII after storage at 25° C./60% relative humidity for 3 months. Not more than 0.3% of each of the amide impurity of Formula I, diastereomer of atorvastatin calcium of Formula II, ketal of atorvastatin of Formula III, 3′-deshydroxy atorvastatin of Formula IV, 5′-deshydroxy atorvastatin of Formula V, methyl ester of atorvastatin of Formula VI, diepoxide of atorvastatin of Formula VII, dihydroxy diepoxide of Formula VIII, O-methyl atorvastatin calcium of Formula X, 3-oxo atorvastatin of Formula XI, unidentified Impurity at RRT˜1.03 of MW 590, or the unidentified impurity at RRT˜1.17 of MW 431 or the corresponding lactone of atorvastatin of Formula XII was found after storage at 25° C./60% relative humidity for 3 months in the amorphous atorvastatin calcium obtained by the processes of the invention.

Amorphous atorvastatin calcium obtained contained not more than 0.15% of each of the amide impurity of Formula I, diastereomer of atorvastatin calcium of Formula II, ketal of atorvastatin of Formula III, 3′-deshydroxy atorvastatin of Formula IV, 5═-deshydroxy atorvastatin of Formula V, methyl ester of atorvastatin of Formula VI, dihydroxy diepoxide of Formula VIII, O-methyl atorvastatin calcium of Formula X, 3-oxo atorvastatin of Formula XI, unidentified Impurity at RRT˜1.03 of MW 590, unidentified impurity at RRT˜1.17 of MW 431 or the corresponding lactone of atorvastatin of Formula XII after storage at 25° C./60% relative humidity for 3 months.

Amorphous atorvastatin calcium containing not more than 0.1% of each of the amide impurity of Formula I, diastereomer of atorvastatin calcium of Formula II, ketal of atorvastatin of Formula III, 3′-deshydroxy atorvastatin of Formula IV, 5′-deshydroxy atorvastatin of Formula V, methyl ester of atorvastatin of Formula VI, dihydroxy diepoxide of Formula VIII, O-methyl atorvastatin calcium of Formula X, or the unidentified impurity at RRT˜1.17 of MW 431 after storage at 25° C./60% relative humidity for 3 months.

Amorphous atorvastatin calcium containing not more than 0.05% of each of amide impurity of Formula I, diastereomer of atorvastatin calcium of Formula II, 3′-deshydroxy atorvastatin of Formula IV, 5′-deshydroxy atorvastatin of Formula V, methyl ester of atorvastatin of Formula VI, dihydroxy diepoxide of Formula VIII, or the O-methyl atorvastatin calcium of Formula X after storage at 25° C./60% relative humidity for 3 months.

Amorphous atorvastatin calcium in general contained not more than 1.0% total impurities and/or degradants after storage at 25° C./60% relative humidity for 3 months.

In the following section embodiments are described by way of example to illustrate the process disclosed herein. However, these do not limit the scope of the present invention.

### EXAMPLE 1

### Preparation of amorphous[R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1) (Atorvastatin Calcium Amorphous)

### (4R-cis)-1,1-dimethylethyl-6-{2-[2-(4-fluorophenyl)-5-(1-methylethyl)-3-phenyl-4-[(phenylamino)-carbonyl]-1H-pyrrol-1yl]ethyl}-2,2-dimethyl-1,3-dioxane-4-acetate (Compound H)

A mixture of (4R-cis)-1,1-dimethylethyl-6-(2-aminoethyl)-2,2-dimethyl-1,3-dioxane]-4-acetate (9 Kg, 32.96 moles), (±)-4-fluoro-α-(2-methyl-1-oxopropyl)-γ-oxo-N,β-diphenylbenzenebutaneamide (13.33 Kg, 31.93 moles), n-heptane (90 L), tetrahydrofuran (22.5 L), toluene (22.5 L) and pivalic acid (2.18 Kg, 21.30 moles) was heated to reflux temperature for about 40 hrs. The reaction was monitored for completion by HPLC. The reaction mass was cooled and diluted with toluene. The reaction mixture was then washed initially with aqueous sodium hydroxide solution (0.5 N), then with aqueous hydrochloric acid solution (0.5 N) and followed by brine (10%). The organic layer was treated with activated carbon, and filtered through a hyflo filter. The organic layer was concentrated to 10% of the total volume under vacuum. Isopropyl alcohol (34 L) was then added, and the solvent recovered under vacuum, followed by repeated addition of isopropyl alcohol and solvent recovery under vacuum. The residue was dissolved in isopropyl alcohol and de-ionized water (45 L) was added till turbidity appeared. Further de-ionized water (60 L) was added gradually. The precipitated product was filtered, washed with a mixture of isopropyl alcohol and de-ionized water (2:1) and dried to get the title compound (16.2 Kg, 24.77 moles, 94% by HPLC). The crude product was purified by dissolving in isopropyl alcohol (128 L) at 50 to 55° C., concentrating the solution and cooling the residual mass slowly under stirring. The solid thus obtained was filtered, washed with chilled isopropyl alcohol and dried at 40 to 45° C. to give pure Compound H (13.2 Kg, 20.20 moles, purity: 99% by HPLC).

### [R—(R*,R*)]-1,1-Dimethylethyl-2-(4-fluorophenyl)-β,δ-dihydroxy-5-(1-methylethyl)-3-phenyl-4-[(phenylamino)carbonyl]-1H-pyrrole-1-heptanoate (Compound I)

To a solution of Compound H (10 Kg, 15.29 moles) in methanol (217 L), 1 N hydrochloric acid solution (21 L, 16.04 moles) was added at 20-26° C. in 15 minutes. The reaction mixture was stirred at the same temperature until the reaction was complete (about 6 hours, monitoring by HPLC).

### [R—(R*,R*)]-2-(4-Fluorophenyl)-β,δ-dihydroxy-5-(1-methylethyl)-3-phenyl-4-[(phenylamino)-carbonyl]-1H-pyrrole-1-heptanoic acid, sodium salt (Atorvastatin Sodium)

The pH of the reaction mixture obtained above was adjusted to about 12 by adding 10% w/v aqueous sodium hydroxide solution at 25-30° C. and the resulting mixture was stirred for about 6 hours at 25-30° C. The progress of the reaction was monitored by HPLC. The pH of the reaction mixture was monitored and maintained at about 12 throughout the course of the reaction by adding 10% w/v aqueous sodium hydroxide solution. After the reaction was complete, the mass was filtered and concentrated to about 84 L.

### Crude [R—(R*,R*)]-2-(4-fluorophenyl)-β,δ-dihydroxy-5-(1-methylethyl)-3-phenyl-4-[(phenylamino)-carbonyl]-1H-pyrrol-1-heptanoic acid, calcium salt (2:1) (Atorvastatin Calcium Crude)

De-ionized water (89 L), methanol (19 L), and methyl tertiary butyl ether (60 L), were added to the concentrated reaction mass obtained above with continuous stirring. The layers were separated. The aqueous layer was washed with methyl tertiary butyl ether and filtered through a sparkler filter. The filtrate was collected in a reactor and its pH adjusted to 7.9-8.1 with 6 N hydrochloric acid. This mixture was heated to 48° C.

To this mixture, an aqueous solution of calcium acetate (1.33 Kg, 8.41 moles) in water (48 L) was added slowly and heated to 51° C. The contents were stirred at 51-54° C. until a clear solution was obtained. Crystalline atorvastatin calcium Form I seeds (77 g) were added and stirred. Methyl tertiary butyl ether was recovered under reduced pressure. The temperature was raised to 58° C. and de-ionized water (11 L) was added. The contents were cooled to 50° C. and a second lot of atorvastatin calcium Form I seeds (33 g) were added. The contents were further cooled slowly to 30° C. over a period of 3-4 hours and filtered. The wet cake was washed with a mixture of methanol and de-ionized water. The material was dried at 45-50° C. to yield 8 Kg of atorvastatin calcium having a purity of 97.5% determined by HPLC.

### Pure Crystalline Atorvastatin Calcium

The crude product obtained above was charged to a reactor containing methanol (16 L) and tetrahydrofuran (40 L). The contents were stirred to get a clear solution and filtered through a sparkler filter followed by washing over hyflo bed with methanol (32 L).

The filtrate was heated to 65° C. and refluxed for 30-60 minutes. To this, de-ionized water (about 120 L) was added slowly over a period of 1-2 hours until turbidity appeared. At the onset of turbidity, crystalline atorvastatin calcium Form I seeds (8 g) were added. The contents were stirred for 30 minutes at 68-72° C. and de-ionized water (about 40 L) was added. The contents were cooled to 50° C. and atorvastatin calcium Form I seeds (24 g) were added with continuous stirring. The contents were further cooled to 35° C. and stirred for 5 hours at 33-35° C. and then filtered. The wet cake was washed with a mixture of tetrahydrofuran, methanol and de-ionized water (volume ratio 1:1:4) and then dried at 50-55° C. under reduced pressure to yield 7.36 Kg of crystalline atorvastatin calcium.

### Preparation of Crystalline Atorvastatin Calcium (Form-I)

The above dried product was added to a reactor containing de-ionized water (108.8 L) and methanol (19.2 L). The contents were stirred for 10 minutes and heated to 45° C. To this, crystalline atorvastatin calcium form I seeds (730 g) were added and the mixture was stirred at 40° C. to 45° C. until the IR spectrum of the sample was comparable with the seed crystals. The contents were filtered and washed with a mixture of de-ionized water and methanol (volume ratio 6:1). The wet cake was dried at 50-55° C. to yield 7.2 Kg of crystalline atorvastatin calcium having a purity of 99.7% determined by HPLC.

### Atorvastatin Calcium Amorphous

Tetrahydrofuran (16.38 L) was added to crystalline atorvastatin calcium Form 1 (6.3 Kg, 5.2 moles) obtained above followed by butylated hydroxyanisole (63 g, 0.5 moles). The contents were stirred for 30 minutes at 20 to 25° C. to get a solution. This solution was filtered over a hyflo bed followed by washing of the hyflo bed with tetrahydrofuran (2.52 L), and the filtrate was collected. The filtrate was added slowly over a period of 4 to 5 hours to cyclohexane (189 L) at 25° C. The contents were stirred for 60 minutes, centrifuged and washed with cyclohexane. The material was dried under vacuum at 60° C. to 70° C. for 12 hours to yield 5.67 Kg of amorphous atorvastatin calcium having a purity of 99.54% determined by HPLC.

### EXAMPLE 2

### Preparation of Atorvastatin Calcium Amorphous

Tetrahydrofuran (10 L) was added to atorvastatin calcium Form I (1 Kg) obtained as per Example 1 above, followed by butylated hydroxyanisole (3 g). The contents were stirred for 15 minutes at 20 to 25° C. to get a solution. This solution was filtered over hyflo bed followed by washing of the hyflo bed with tetrahydrofuran (0.4 L), and the filtrate was collected and concentrated to a volume of about 3 L at 62 to 70° C. The solution was cooled to 20° C. and added slowly over a period of 4 to 5 hours to cyclohexane (30 L) at 20 to 23° C. The contents were stirred for 60 minutes and filtered. The wet cake was washed with cyclohexane. The material was dried under vacuum at 60° C. to 70° C. for 12 hours to yield 0.9 Kg of amorphous atorvastatin calcium having a purity of 99.45% determined by HPLC.

### Preparation of Crystalline Atorvastatin Calcium (Form-II)

### EXAMPLE 3

A mixture of methanol (180 ml) and de-ionized water (120 ml) was added to crystalline atorvastatin calcium form I (15 g) at room temperature. The temperature was raised to 25° C., seeds of crystalline atorvastatin calcium form II (1.5 g) were added, and the suspension was stirred at 25° C. The suspension became very thick after about 24 hours and a mixture of methanol (90 ml) and de-ionized water (60 ml) was added to resume stirring. The suspension was further stirred at 25° C. for another 24 hours and then filtered. The filtered solids were dried under reduced pressure at 70° C. for 48 hours to get 14.7 g of crystalline atorvastatin calcium. The XRD spectrum of the product matched with that of Form-II of atorvastatin calcium.

### EXAMPLE 4

A mixture of methanol (1.2 L) and de-ionized water (800 ml) was added to crystalline atorvastatin calcium Form I (100 g) at room temperature. The temperature was raised to 45° C. slowly, seeds of crystalline atorvastatin calcium form II (10 g) were added, and the suspension stirred at 45° C. The suspension became very thick after about 24 hours and a mixture of methanol (600 ml) and de-ionized water (400 ml) was added to resume stirring. The suspension was again warmed to 45° C. and further stirred at the same temperature for another 24 hours and then filtered. The filtered solids were dried under reduced pressure at 70° C. for 48 hours to get 98 g of crystalline atorvastatin calcium. The XRD spectrum of the product matched with that of Form-II of atorvastatin calcium.

### EXAMPLE 5

### Purification of Atorvastatin Calcium (Without Seeding)

The crude atorvastatin calcium obtained as per Example 1 was charged to a reactor containing methanol (16 L) and tetrahydrofuran (40 L). The contents are stirred to get a clear solution and filtered through a sparkler filter, followed by washing over hyflo bed with methanol (32 L). The filtrate was heated to 65° C. and refluxed for 30-60 minutes. To this, de-ionized water (about 120 L) was added slowly over a period of 1-2 hours until turbidity appeared. The contents were stirred for 30 minutes at 68-72° C. and de-ionized water (about 40 L) was added. The contents were cooled to 35° C. and stirred for 5 hours at 33-35° C. and then filtered. The wet cake was washed with a mixture of tetrahydrofuran, methanol and de-ionized water (volume ratio 1:1:4) and then dried at 50-55° C. under reduced pressure to yield 7.33 Kg of crystalline atorvastatin calcium.

### EXAMPLE 6

### Preparation of Atorvastatin Calcium Amorphous

Tetrahydrofuran (480 ml) was added to crystalline atorvastatin calcium obtained above in Example 5 (60 g), followed by butylated hydroxyanisole (0.6 g). The contents were stirred, de-ionized water (24 ml) was added and the mixture was stirred for 15 minutes at 20 to 25° C. to get a clear solution. Molecular sieves (240 g, Siliporite NK30 AP® powdered) were added to the solution and the mixture was stirred for 2 hours at 20 to 25° C. This solution was filtered through a molecular sieves bed, followed by washing of the bed with tetrahydrofuran (120 ml). The filtrate was collected and concentrated to a volume of about 210 ml at 60 to 70° C. The concentrated solution was cooled to 25° C. and added slowly over a period of 2 hours to cyclohexane (1800 ml) at 22 to 25° C. under moderate stirring. The contents were stirred vigorously for 30 minutes at the same temperature and filtered. The wet cake was washed with cyclohexane (60 ml). The material was dried under reduced pressure at 60° C. to 70° C. for 6 hours to yield 54 g of amorphous atorvastatin calcium. Siliporite NK30 AP is registered trademark of CECA, France

### EXAMPLE 7

### Preparation of Crystalline Atorvastatin Calcium (Form II)

A mixture of methanol (1.2 L) and de-ionized water (800 ml) was added to crystalline atorvastatin calcium obtained above in example 5 (100 g) at room temperature. The temperature was raised to 45° C. slowly, seeds of crystalline atorvastatin calcium form II (10 g) were added, and the suspension stirred at 45° C. The suspension became very thick after about 24 hours and a mixture of methanol (600 ml) and de-ionized water (400 ml) was added to resume stirring. The suspension was again warmed to 45° C. and further stirred at the same temperature for another 24 hours and then filtered. The filtered solids were dried under reduced pressure at 70° C. for 48 hours to get 98 g of crystalline atorvastatin calcium. The XRD spectrum of the product matched with that of Form-II of atorvastatin calcium.

### EXAMPLE 8

### Preparation of Atorvastatin Calcium Amorphous

Tetrahydrofuran (10 L) was added to atorvastatin calcium crystalline Form II (1 Kg) followed by butylated hydroxyanisole (3 g). The contents were stirred for 15 minutes at 20 to 25° C. to get a solution. This solution was filtered over a hyflo bed followed by washing of the hyflo bed with tetrahydrofuran (0.4 L), and the filtrate was collected and concentrated to a volume of about 3 L at 62 to 70° C. The solution was cooled to 20° C. and added slowly over a period of 4 to 5 hours to cyclohexane (30 L) at 20 to 23° C. The contents were stirred for 60 minutes and filtered. The wet cake was washed with cyclohexane. The material was dried under vacuum at 60° C. to 70° C. for 12 hours to yield 0.9 Kg of amorphous atorvastatin calcium having a purity of 99.5% determined by HPLC.

### EXAMPLE 9

### Atorvastatin Calcium Amorphous

Methanol (100 mL) was added to atorvastatin calcium form II (10 g). The contents were stirred for 40 minutes at 20 to 25° C. to get a clear solution. Butylated hydroxyanisole (0.1 g) was then added and the mixture stirred for 30 minutes. Methanol (50 ml) was then recovered at 40° C. under reduced pressure in 30 minutes. The solution was cooled to 20 to 25° C. and added slowly over a period of one hour to cyclohexane (300 mL) at 20 to 30° C. The solution was stirred for 1 hour at 25° C. The obtained clear solution was concentrated to a volume of about 300 L at 60 to 70° C. (approximately 50 ml methanol was distilled out). The obtained suspension was then cooled to 20 to 25° C. with stirring in 30 minutes and filtered. The wet cake was washed with cyclohexane. The material was dried under vacuum at 60° C. to 70° C. for 1 hour to yield 9.0 g of amorphous atorvastatin calcium.

### EXAMPLE 10

### Preparation of (4R-cis)-1,1-dimethylethyl-[6-(2-aminoethyl)-2,2-dimethyl-1,3-dioxan-4-yl]acetate (Compound F)

### (R)-1,1-dimethylethyl-6-cyano-5-hydroxy-3-oxohexanoate (Compound C)

n-Butyl lithium and diisopropylamine are mixed at −40° C. for the formation of lithium diisopropylamine. 1,1-Dimethylethyl acetate (Compound B) in tetrahydrofuran is then added at −50° C. and the mixture stirred at −20 to 25° C. for 1 hour. The reaction mixture is further cooled to −50° C. and (R)-ethyl-4-cyano-3-hydroxybutanoate is added maintaining temperature at −20 to −25° C. and kept at the same temperature for 2 hours. The reaction is monitored for completion by thin layer chromatography. The reaction is quenched with a 2 N solution of hydrochloric acid and the product was extracted with ethyl acetate, washed with water and brine followed by complete evaporation of solvent to get the title compound which is taken to the next step without further purification.

### [R—(R*,R*)]-1,1-dimethylethyl 6-cyano-3,5-dihydroxyhexanoate Compound D)

To a mixture of tetrahydrofuran and methanol containing (R)-1,1-dimethylethyl-6-cyano-5-hydroxy-3-oxohexanoate (Compound C) is added diethyl methoxyborane slowly at −80 to −90° C. and stirred for 30 minutes at the same temperature. Sodium borohydride is added in lots maintaining temperature at −80 to −90° C. and stirred for 5 hours at −80 to −90° C. After completion of reaction, the temperature is slowly raised to 0° C. then to room temperature in 2 hours, and the reaction quenched with glacial acetic acid slowly in 30 minute while maintaining temperature between 0 to 40° C. The mixture is concentrated to approximately 20% of total volume. Methanol is added and recovered to remove borane derivatives. The product is extracted with ethyl acetate, washed with water and then brine. The organic layer is concentrated to approximately 20% of original volume. Tetrahydrofuran is then added and recovered completely under reduced pressure to get the title compound as a concentrated mass, which is taken to the next step.

### (4R-cis)-1,1-Dimethylethyl-[6-cyanomethyl-2,2-dimethyl-1,3-dioxan]-4-acetate (Compound E)

A mixture of 2,2-dimethoxy propane containing [R—(R*,R*)]-1,1-dimethylethyl-6-cyano-3,5-dihydroxyhexanoate (Compound D), acetone and methanesulfonic acid is stirred for 3 to 4 hours at 29 to 30° C. and the reaction monitored for completion by thin layer chromatography. The reaction is then quenched with 5% w/v aqueous sodium bicarbonate solution slowly to adjust pH to about 7 and extracted with ethyl acetate. The organic layer is concentrated and the solvent recovered completely under reduced pressure. The residue is crystallized with hexane to get the title compound as a crude product, which is recrystallized with methanol and water to get the pure compound.

### (4R-cis)-1,1-Dimethylethyl-[6-(2-aminoethyl)-2,2-dimethyl-1,3-dioxan-4-yl]acetate (Compound F)

Pure (4R-cis)-1,1-dimethylethyl-[6-cyanomethyl-2,2-dimethyl-1,3-dioxan]-4-acetate (Compound E) dissolved in ammonia saturated methanol is hydrogenated in the presence of activated Raney nickel by applying hydrogen pressure of 4.5 to 5 kg/cm² at room temperature under stirring for 4 to 12 hours. The reaction is monitored for completion by gas chromatography. The catalyst is filtered through hyflo bed and concentrated to recover methanol completely under reduced pressure to get the title compound.

### EXAMPLE 11

### Preparation of Crystalline Atorvastatin Calcium Form-I Seed

### Part A—Preparation of Crude Atorvastatin Calcium

### [R—(R*,R*)]-1,1-Dimethylethyl-2-(4-fluorophenyl)-β,δ-dihydroxy-5-(1-methylethyl)-3-phenyl-4-[(phenylamino)carbonyl]-1H-pyrrole-1-heptanoate (Compound I)

To a solution of Compound H (57 g) in methanol (1.71 L), 1 N hydrochloric acid solution (116 mL) was added drop wise at 20-25° C. in 15 minutes. The reaction mixture was stirred at the same temperature for about 5 hours, and monitored by TLC (hexane:ethanol::6:4). 1 N hydrochloric acid solution (10 mL) was then added and the reaction mixture was further stirred for about 2.5 hours.

### [R—(R*,R*)]-2-(4-Fluorophenyl)-β,δ-dihydroxy-5-(1-methylethyl)-3-phenyl-4-[(phenylamino)-carbonyl]-1H-pyrrole-1-heptanoic acid, sodium salt (Atorvastatin Sodium)

The pH of the reaction mixture obtained above was adjusted to about 12 by adding 10% w/v aqueous sodium hydroxide solution at 25-30° C. and the resulting mixture was stirred for about 6 hours at 25-30° C. The progress of the reaction was monitored by HPLC. The pH of the reaction mixture was monitored and maintained at about 12 throughout the course of the reaction by adding 10% w/v aqueous sodium hydroxide solution. After the reaction was complete, the mass was filtered and concentrated to get the title compound as a white precipitate.

### [R—(R*,R*)]-2-(4-fluorophenyl)-β,δ-dihydroxy-5-(1-methylethyl)-3-phenyl-4-[(phenylamino)-carbonyl]-1H-pyrrol-1-heptanoic acid, calcium salt (2:1) (Atorvastatin Calcium)

De-ionized water (500 mL), methanol (200 mL), and methyl tertiary butyl ether (200 mL), were added to atorvastatin sodium obtained above and stirred to obtain a solution. The layers were separated. The aqueous layer was washed with methyl tertiary butyl ether (200 mL), and filtered through hyflo bed. The hyflo bed was washed with a mixture of methanol (25 mL) and methyl tertiary butyl ether (25 mL). The filtrate was warmed to 50° C. and its pH adjusted to about 10 with concentrated hydrochloric acid. To this mixture, an aqueous solution of calcium acetate (7.52 g) in water (275 mL) was added slowly in 1.5 hours at 50° C. Some turbidity was observed at this stage. Methyl tertiary butyl ether (20 mL) was added. Some methyl tertiary butyl ether spontaneously evaporated at this stage. The mixture was heated to 80° C. to obtain a clear solution. The contents were stirred for 20 minutes at the same temperature and then allowed to cool for 1.5 hours. The contents were further cooled to 25° C. and stirred for 30 minutes at the same temperature and then filtered. The wet product was slurry washed with a mixture of methanol and de-ionized water (2:1, 100 mL) and filtered. The material was dried at 45° C. for 8 hours to yield 43.38 g of atorvastatin calcium. The XRD spectrum of the product mainly showed two very broad peaks.

### Part B—Preparation of Crystalline Atorvastatin Calcium Form-I Seed

### Step I

Atorvastatin calcium (2 g) obtained above was suspended in de-ionized water (20 ml) and stirred for 20 hours at about 30° C. The suspension was then filtered and dried under reduced pressure at 40 to 45° C. for 3 hours to get 1.9 g of the product. The XRD spectrum of the product showed a change in pattern from that of the starting atorvastatin calcium. An increase in sharp peaks indicating increased crystallinity was observed.

### Step II

Atorvastatin calcium (1.8 g, same as that used as starting compound in step I) and atorvastatin calcium (0.2 g, obtained from step I above) were suspended in a mixture of de-ionized water (34 ml) and methanol (6 ml). The temperature was raised slowly to 38 to 40° C. and the suspension was stirred for 16 hours at the same temperature. The suspension was then cooled to 35° C., filtered and dried under reduced pressure at 40 to 45° C. for 4 hours to get 1.9 g of the product. The XRD spectrum of the product showed a change in pattern from that of the starting atorvastatin calcium. The XRD spectrum of the product matched with that of Form-I of atorvastatin calcium.

### EXAMPLE 12

### Preparation of Crystalline Atorvastatin Calcium Form-II Seed

A mixture of methanol (360 ml) and de-ionized water (240 ml) was added to a mixture of amorphous atorvastatin calcium (15 g) and crystalline atorvastatin calcium form I (15 g), the suspension was warmed to 45° C. slowly and stirred at the same temperature. The suspension became very thick after 24 hours and a mixture of methanol (180 ml) and de-ionized water (120 ml) was added to resume stirring. The suspension was warmed to 45° C. and further stirred at the same temperature for 24 hours and then filtered. The filtered solids were dried under reduced pressure at 70° C. for 48 hours to get 27 g of crystalline atorvastatin calcium. The XRD spectrum of the product matched with that of Form-II of atorvastatin calcium.

1. Amorphous atorvastatin calcium containing not more than 0.2% of a compound selected from the group consisting of amide impurity of Formula I, diastereomer of atorvastatin calcium of Formula II, ketal of atorvastatin of Formula III, 3′-deshydroxy atorvastatin of Formula IV, 5′-deshydroxy atorvastatin of Formula V, methyl ester of atorvastatin of Formula VI, diepoxide of atorvastatin of Formula VII, dihydroxy diepoxide of Formula VIII, diketoepoxide of Formula IX, O-methyl atorvastatin calcium of Formula X, 3-oxo atorvastatin of Formula XI, unidentified Impurity at RRT˜1.03 of MW 590, unidentified impurity at RRT˜1.17 of MW 431, corresponding lactone of atorvastatin of Formula XII and mixtures thereof.

2. Amorphous atorvastatin calcium containing not more than 0.1% of a compound selected from the group consisting of amide impurity of Formula I, diastereomer of atorvastatin calcium of Formula II, ketal of atorvastatin of Formula III, 3′-deshydroxy atorvastatin of Formula IV, 5′-deshydroxy atorvastatin of Formula V, methyl ester of atorvastatin of Formula VI, diepoxide of atorvastatin of Formula VII, dihydroxy diepoxide of Formula VIII, diketoepoxide of Formula IX, O-methyl atorvastatin calcium of Formula X, 3-oxo atorvastatin of Formula XI, unidentified Impurity at RRT˜1.03 of MW 590, unidentified impurity at RRT˜1.17 of MW 431, corresponding lactone of atorvastatin of Formula XII and mixtures thereof.

3. Amorphous atorvastatin calcium containing not more than 0.05% of a compound selected from the group consisting of amide impurity of Formula I, diastereomer of atorvastatin calcium of Formula II, 3′-deshydroxy atorvastatin of Formula IV, 5′-deshydroxy atorvastatin of Formula V, methyl ester of atorvastatin of Formula VI, diepoxide of atorvastatin of Formula VII, dihydroxy diepoxide of Formula VIII, diketoepoxide of Formula IX, O-methyl atorvastatin calcium of Formula X, unidentified Impurity at RRT˜1.03 of MW 590, unidentified impurity at RRT˜1.17 of MW 431 and mixtures thereof.

4. Amorphous atorvastatin calcium according to claims 1 to 3 containing not more than 0.5% of total impurities.

5. Amorphous atorvastatin calcium containing not more than 0.5% of a compound selected from the group consisting of amide impurity of Formula I, diastereomer of atorvastatin calcium of Formula II, ketal of atorvastatin of Formula III, 3′-deshydroxy atorvastatin of Formula IV, 5′-deshydroxy atorvastatin of Formula V, methyl ester of atorvastatin of Formula VI, diepoxide of atorvastatin of Formula VII, dihydroxy diepoxide of Formula VIII, diketoepoxide of Formula IX, O-methyl atorvastatin calcium of Formula X, 3-oxo atorvastatin of Formula XI, unidentified Impurity at RRT˜1.03 of MW 590, unidentified impurity at RRT˜1.17 of MW 431, corresponding lactone of atorvastatin of Formula XII and mixtures thereof after storage at 25° C./60% relative humidity for 3 months.

6. Amorphous atorvastatin calcium containing not more than 0.3% of a compound selected from the group consisting of amide impurity of Formula I, diastereomer of atorvastatin calcium of Formula II, ketal of atorvastatin of Formula III, 3′-deshydroxy atorvastatin of Formula IV, 5′-deshydroxy atorvastatin of Formula V, methyl ester of atorvastatin of Formula VI, diepoxide of atorvastatin of Formula VII, dihydroxy diepoxide of Formula VIII, O-methyl atorvastatin calcium of Formula X, 3-oxo atorvastatin of Formula XI, unidentified Impurity at RRT˜1.03 of MW 590, unidentified impurity at RRT˜1.17 of MW 431, corresponding lactone of atorvastatin of Formula XII and mixtures thereof after storage at 25° C./60% relative humidity for 3 months.

7. Amorphous atorvastatin calcium containing not more than 0.15% of a compound selected from the group consisting of amide impurity of Formula I, diastereomer of atorvastatin calcium of Formula II, ketal of atorvastatin of Formula III, 3′-deshydroxy atorvastatin of Formula IV, 5′-deshydroxy atorvastatin of Formula V, methyl ester of atorvastatin of Formula VI, dihydroxy diepoxide of Formula VIII, O-methyl atorvastatin calcium of Formula X, 3-oxo atorvastatin of Formula XI, unidentified Impurity at RRT˜1.03 of MW 590, unidentified impurity at RRT˜1.17 of MW 431, corresponding lactone of atorvastatin of Formula XII and mixtures thereof after storage at 25° C./60% relative humidity for 3 months.

8. Amorphous atorvastatin calcium containing not more than 0.1% of a compound selected from the group consisting of amide impurity of Formula I, diastereomer of atorvastatin calcium of Formula II, ketal of atorvastatin of Formula III, 3′-deshydroxy atorvastatin of Formula IV, 5′-deshydroxy atorvastatin of Formula V, methyl ester of atorvastatin of Formula VI, dihydroxy diepoxide of Formula VIII, O-methyl atorvastatin calcium of Formula X, unidentified impurity at RRT˜1.17 of MW 431 and mixtures thereof after storage at 25° C./60% relative humidity for 3 months.

9. Amorphous atorvastatin calcium containing not more than 0.05% of a compound selected from the group consisting of amide impurity of Formula I, diastereomer of atorvastatin calcium of Formula II, 3′-deshydroxy atorvastatin of Formula IV, 5′-deshydroxy atorvastatin of Formula V, methyl ester of atorvastatin of Formula VI, dihydroxy diepoxide of Formula VIII, O-methyl atorvastatin calcium of Formula X and mixtures thereof after storage at 25° C./60% relative humidity for 3 months.

10. Amorphous atorvastatin calcium according to claims 5 to 9 containing not more than 1.0% total impurities and/or degradants after storage at 25° C./60% relative humidity for 3 months.
